SEQUENCE LISTING (27 sequences)

SEQ ID NO: 1 - protein, 8 aa, Artificial Sequence
  source: /note=“Description of Artificial Sequence Synthetic peptide”
GFTLDYYG

SEQ ID NO: 2 - protein, 8 aa, Artificial Sequence
  source: /note=“Description of Artificial Sequence Synthetic peptide”
ISASARTI

SEQ ID NO: 3 - protein, 21 aa, Artificial Sequence
  source: /note=“Description of Artificial Sequence Synthetic peptide”
ARRRFSASSVNRWLADDYDVW

SEQ ID NO: 4 - protein, 8 aa, Artificial Sequence
  source: /note=“Description of Artificial Sequence Synthetic peptide”
GSIAGFET

SEQ ID NO: 5 - protein, 8 aa, Artificial Sequence
  source: /note=“Description of Artificial Sequence Synthetic peptide”
GFSLDYYG

SEQ ID NO: 6 - protein, 10 aa, Artificial Sequence
  source: /note=“Description of Artificial Sequence Synthetic peptide”
SERNPERNPG

SEQ ID NO: 7 - protein, 8 aa, Artificial Sequence
  source: /note=“Description of Artificial Sequence Synthetic peptide”
GSSFSTST

SEQ ID NO: 8 - protein, 7 aa, Artificial Sequence
  source: /note=“Description of Artificial Sequence Synthetic peptide”
QSVSSSY

SEQ ID NO: 9 - protein, 8 aa, Artificial Sequence
  source: /note=“Description of Artificial Sequence Synthetic peptide”
GYSFTSYW

SEQ ID NO: 10 - protein, 8 aa, Artificial Sequence
  source: /note=“Description of Artificial Sequence Synthetic peptide”
MTKTNNEI

SEQ ID NO: 11 - protein, 8 aa, Artificial Sequence
  source: /note=“Description of Artificial Sequence Synthetic peptide”
ISASAKTK

SEQ ID NO: 12 - protein, 8 aa, Artificial Sequence
  source: /note=“Description of Artificial Sequence Synthetic peptide”
WQTGGSLN

SEQ ID NO: 13 - protein, 7 aa, Artificial Sequence
  source: /note=“Description of Artificial Sequence Synthetic peptide”
FTSGGAI

SEQ ID NO: 14 - protein, 8 aa, Artificial Sequence
  source: /note=“Description of Artificial Sequence Synthetic peptide”
FYPGDSST

SEQ ID NO: 15 - protein, 6 aa, Artificial Sequence
  source: /note=“Description of Artificial Sequence Synthetic peptide”
KGPELR

SEQ ID NO: 16 - protein, 13 aa, Artificial Sequence
  source: /note=“Description of Artificial Sequence Synthetic peptide”
ARRRFDASASNRW

SEQ ID NO: 17 - protein, 10 aa, Artificial Sequence
  source: /note=“Description of Artificial Sequence Synthetic peptide”
YLKKWRDQYW

SEQ ID NO: 18 - protein, 11 aa, Artificial Sequence
  source: /note=“Description of Artificial Sequence Synthetic peptide”
ALHNAVSGSSW

SEQ ID NO: 19 - protein, 10 aa, Artificial Sequence
  source: /note=“Description of Artificial Sequence Synthetic peptide”
QQYGSSTWTF

SEQ ID NO: 20 - protein, 13 aa, Artificial Sequence
  source: /note=“Description of Artificial Sequence Synthetic peptide”
ARRRNWGNAFDIW

SEQ ID NO: 21 - protein, 17 aa, Artificial Sequence
  source: /note=“Description of Artificial Sequence Synthetic peptide”
AKSKPGYCGGGSCSFDS

SEQ ID NO: 22 - protein, 9 aa, Artificial Sequence
  source: /note=“Description of Artificial Sequence Synthetic peptide”
QQYGSSPLT

SEQ ID NO: 23 - protein, 11 aa, Artificial Sequence
  source: /note=“Description of Artificial Sequence Synthetic peptide”
ARIARINGRTW

SEQ ID NO: 24 - protein, 9 aa, Artificial Sequence
  source: /note=“Description of Artificial Sequence Synthetic peptide”
QQYDSYSWT

SEQ ID NO: 25 - protein, 9 aa, Artificial Sequence
  source: /note=“Description of Artificial Sequence Synthetic peptide”
QQYDHYSWT

SEQ ID NO: 26 - protein, 13 aa, Artificial Sequence
  source: /note=“Description of Artificial Sequence Synthetic peptide”
AKVLGSWSGAMDV

SEQ ID NO: 27 - protein, 9 aa, Artificial Sequence
  source: /note=“Description of Artificial Sequence Synthetic peptide”
QQYVSSPRA

(19) United States
(12) Patent Application Publication
(10) Pub. No.: US 2024/0011985 A1
(43) Pub. Date: Jan. 11, 2024

(54) NEUTRALIZED ANTIBODY AND METHOD OF USE THEREOF

(71) Applicant: FZata, Inc., Halethorpe (MD)

(72) Inventors: Zhiyong Yang, West Friendship, MD (US); Hua Yu, Columbia, MD (US)

(73) Assignee: FZata, Inc., Halethorpe, MD (US)

(21) Appl. No.: 18/032,657

(22) PCT Filed: Oct. 19, 2021
(86) PCT No.: PCT/US2021/055686

§ 371 Date: Apr. 19, 2023

Related U.S. Application Data
(60) Provisional application No. 63/093,884, filed on Oct. 20, 2020.

(51) Int. Cl. G01N 33/569

(52) U.S. Cl. CPC G01N 33/56911; G01N 2333/33; G01N 2469/20; G01N 2470/12

(57) ABSTRACT

Described herein, are methods of detecting neutralizing antibodies that bind to Clostridioides difficile (C. diff) toxin B (TcdB), prognosing the disease severity of Clostridioides difficile infection (CDI) and the risk of primary and recurrent CDI, as well as providing a guide for clinical practice. Also described herein are kits for performing the methods of this disclosure.

### CROSS-REFERENCE TO RELATED PATENT APPLICATIONS

This application is a U.S. National Stage Application under 35 U.S.C. § 371 of International Patent Application No. PCT/US2021/055686 filed Oct. 19, 2021, which claims priority under U.S.C. § 119(e) to U.S. Provisional Patent Application No. 63/093,884, filed Oct. 20, 2020, the entire contents of which are incorporated herein by reference.

### GOVERNMENT SUPPORT

This invention was made with government support under Grant Nos 1R43AI136176-01 and 2R44AI136176-02 awarded by the National Institutes of Health (NIH). The United States government has certain rights in the invention.

### SEQUENCE LISTING

The instant application contains a Sequence Listing which has been submitted electronically in ASCII format and is hereby incorporated by reference in its entirety. Said ASCII copy, created on Jan. 18, 2022, is named 130507-0105_SL.txt and is 7,159 bytes in size.

### FIELD OF INVENTION

Described herein, are methods of detecting neutralizing antibodies that bind to Clostridioides difficile (C. diff) toxin B (TcdB), prognosing the disease severity of Clostridioides difficile infection (CDI) and the risks of primary and recurrent CDI, and providing a guide for clinical practice for treating CDI. Also described herein are kits for performing the methods of this disclosure.

### BACKGROUND

The following discussion is merely provided to aid the reader in understanding the disclosure and is not admitted to describe or constitute prior art thereto.

Clostridioides difficile (C. diff.)-mediated disease has a complicated pathogenesis, disease manifestation, and appearance. C. diff. is the most common cause of nosocomial antibiotic-associated diarrhea and the etiologic agent of pseudomembranous colitis. The infection causes a range of diseases (collectively designated as C. diff. infection [CDI]) and it is estimated that over 500,000 cases of C. diff.-associated disease occur annually in the US with the annual mortality rate ranging from 3-17% depending on the infecting strain. With the emergence of hypervirulent and antibiotic-resistant strains, the incidence of mortality in CDI patients is increasing rapidly. Antibiotic-resistant C. diff. is responsible for more than 29,000 deaths in the US each year and the infection is ranked as an urgent threat by the CDC.

The clinical outcomes of C. diff. colonization and infection are varied and include asymptomatic carriage, mild self-limiting diarrhea, severe life-threatening pseudomembranous fulminant colitis and death, which are determined by bacterial and host factors such as age, and immune status. Systemic symptoms such as fever, hypotension, nausea, anorexia, and malaise are frequently seen in moderate or severe disease but may be absent in mild disease. A successful management of CDI requires an accurate diagnosis of infection early in the disease course and host protective immunity, and the proper disease severity classification, which all impact the utilization of antimicrobial therapy. The Society for Healthcare Epidemiology of America (SHEA) and the Infectious Diseases Society of America (IDSA) have designed new clinical practice guidelines to improve the diagnosis and management of CDI.

CDI recurrence is the most significant issue faced by physicians as it is difficult to treat and causes lengthy hospitalization and significant financial losses. Current standard treatment for CDI with antibiotics causes the disruption of the microbiota and results in a recurrence rate approaching 35%. The risk of further episodes of CDI in recurrent patients can be more than 50% and a subset of patients will have multiple recurrences. It is a frustrating condition that is difficult to manage and may affect patients for months or even years, causing tremendous morbidity and mortality.

Measuring protective immune responses in patients with CDI is crucial for optimal clinical management and improved prognosis. However, current assays to measure patient toxin-neutralizing antibodies (antitoxins) are based on serum inhibition of toxins' biological activities on cultured cells. This assay needs to specially equipment such as biosafety cabinet, cell culture incubator and microscope. The assay is also subjective as it relies on trained technician to differentiate and count the rounding cells. Moreover, such assays are time consuming, laborious, and difficult to standardize.

### SUMMARY OF THE INVENTION

The present disclosure relates generally to neutralizing antibodies (NAb) and methods of use thereof as well as kits for detection of neutralizing antibodies. More particularly, the present disclosure relates to neutralizing anti-C. diff. toxin B (TcdB) antibodies, and method of use thereof, for prognosis and diagnosis of Clostridioides difficile infection (CDI) and guidance of treatment, as well as kits for detection of NAb against C. diff. TcdB.

In one aspect, the present disclosure provides methods of detecting antibodies that bind to Clostridioides difficile (C. diff) toxin B (TcdB) in a biological sample, comprising: (a) contacting a substrate to which TcdB is attached with (i) a biological sample and (ii) a labeled antibody that binds to TcdB, (b) washing the substrate, and (c) detecting a signal from the labeled antibody, wherein the amount of the signal detected is inversely correlated to the amount of antibodies that binds to TcdB in the sample; wherein the biological sample was obtained from a subject that had or potentially will have a Clostridioides difficile infection (CDI) prior to obtaining the sample.

In some embodiments, the amount of signal is correlated against a predetermined threshold. In some embodiments, the predetermined threshold is determined by an aggregate of clinical samples. For example, the aggregate of clinical samples may comprise about 50 clinical samples, about 100 clinical samples, about 200 clinical, about 300 clinical, about 300 clinical, about 400 clinical, about 500 clinical, about 600 clinical, about 700 clinical, about 800 clinical samples, about 900 clinical, or about 1000 or more clinical samples.

In some embodiments, the labeled antibody is a neutralizing anti-TcdB antibody. For example, the labeled antibody may be selected from ZINPLAVA™ (bezlotoxumab)), N2-IgG, N3-IgG, N11-IgG, 2D-IgG, 2Ds-IgG, 5D-IgG, E3-IgG, 7F-IgG, B7-IgG, B12-IgG, or BB-IgG.

In some embodiments, the antibody that binds to TcdB in the sample is a neutralizing anti-TcdB antibody.

In some embodiments, the biological sample is selected from whole blood, isolated blood cells, plasma, serum, feces, or urine.

In some embodiments, the labeled antibody comprises a label selected from a tag, a fluorophore, an enzyme, a gold particle, a magnetic particle, a dye, or a radiolabel/isotope. In some embodiments, the tag can be biotin or epitope tag.

In some embodiments, the signal is selected from fluorescence, electrochemical, chemiluminescence, or bioluminescence.

In another aspect, the present disclosure provides methods of prognosing Clostridioides difficile infection (CDI), comprising: (a) contacting a substrate to which Clostridioides difficile (C. diff) toxin B (TcdB) is attached with (i) a biological sample and (ii) a labeled antibody that binds to TcdB, (b) washing the substrate, and (c) detecting a signal from the labeled antibody, wherein a signal intensity that is below a predetermined threshold is indicative of a primary CDI or a risk of recurrent CDI, and a signal intensity that is above the predetermined threshold is indicative of lack of primary CDI or a low risk of recurrent CDI; wherein the biological sample was obtained from a subject that had or has a Clostridioides difficile infection (CDI).

In some embodiments, the subject has or had a primary CDI infection. In some embodiments, the subject has or had recurrent CDI infections.

In some embodiments, the predetermined threshold is determined by an aggregate of clinical samples. For example, the aggregate of clinical samples may comprise about 50 clinical samples, about 100 clinical samples, about 200 clinical, about 300 clinical, about 300 clinical, about 400 clinical, about 500 clinical, about 600 clinical, about 700 clinical, about 800 clinical samples, about 900 clinical, or about 1000 or more clinical samples.

In some embodiments, the labeled antibody is a neutralizing anti-TcdB antibody. For example, the labeled antibody may be selected from ZINPLAVA™ (bezlotoxumab)) N2-IgG, N3-IgG, N11-IgG, 2D-IgG, 2Ds-IgG, 5D-IgG, E3-IgG, 7F-IgG, B7-IgG, B12-IgG, or BB-IgG.

In some embodiments, the antibody that binds to TcdB in the sample is a neutralizing anti-TcdB antibody.

In some embodiments, the biological sample is selected from whole blood, isolated blood cells, plasma, serum, feces, or urine.

In some embodiments, the labeled antibody comprises a label selected from a tag, a fluorophore, an enzyme, a gold particle, a magnetic particle, a dye, or a radiolabel/isotope. In some embodiments, the tag can be biotin or epitope tag.

In some embodiments, the signal is selected from fluorescence, electrochemical, chemiluminescence, or bioluminescence.

In another aspect, the present disclosure provides methods of determining likelihood of Clostridioides difficile infection (CDI) and its recurrence , comprising: (a) contacting a substrate to which Clostridioides difficile (C. diff) toxin B (TcdB) is attached with (i) a biological sample and (ii) a labeled antibody that binds to TcdB, (b) washing the substrate, and (c) detecting a signal from the labeled antibody, wherein a signal intensity that is below a predetermined threshold is indicative of CDI (primary or recurrent) or a risk thereof; wherein the biological sample was obtained from a subject that had or has a Clostridioides difficile infection (CDI).

In some embodiments, the subject has or had a primary CDI infection. In some embodiments, the subject has or had recurrent CDI infections.

In some embodiments, the predetermined threshold is determined by an aggregate of clinical samples. For example, the aggregate of clinical samples may comprise about 50 clinical samples, about 100 clinical samples, about 200 clinical, about 300 clinical, about 300 clinical, about 400 clinical, about 500 clinical, about 600 clinical, about 700 clinical, about 800 clinical samples, about 900 clinical, or about 1000 or more clinical samples.

In some embodiments, the labeled antibody is a neutralizing anti-TcdB antibody. For example, the labeled antibody may be selected from ZINPLAVA™ (bezlotoxumab)) N2-IgG, N3-IgG, N11-IgG, C6-IgG, C12-IgG, 2D-IgG, 5D-IgG, E3-IgG, 7F-IgG, A1-IgG, A11-IgG, B7-IgG, B12-IgG, or BB-IgG.

In some embodiments, the antibody that binds to TcdB in the sample is a neutralizing anti-TcdB antibody.

In some embodiments, the biological sample is selected from whole blood, isolated blood cells, plasma, serum, feces, or urine.

In some embodiments, the labeled antibody comprises a label selected from a tag, a fluorophore, an enzyme, a gold particle, a magnetic particle, a dye, or a radiolabel/isotope. In some embodiments, the tag can be biotin or epitope tag.

In some embodiments, the signal is selected from fluorescence, electrochemical, chemiluminescence, or bioluminescence. In another aspect, the present disclosure provides methods for guiding treatment of CDI. For example, in one aspect, the present disclosure provides methods of treating Clostridioides difficile infection (CDI) in a subject, comprising: (a) obtaining a biological sample, wherein the biological sample was obtained from a subject that had a Clostridioides difficile infection (CDI) prior to obtaining the sample, (b) contacting a substrate to which TcdB is attached with (i) the biological sample and (ii) a labeled antibody that binds to TcdB, (c) washing the substrate, (d) detecting a signal from the labeled antibody, wherein the amount of the signal is inversely correlated to primary CDI or development recurrent CDI, and (e) treating the subject with a therapeutic for CDI if the amount of the signal is below a predetermined threshold. In another aspect, the present disclosure provides methods of guiding treatment for a Clostridioides difficile infection (CDI) in a subject, comprising: (a) obtaining a biological sample, wherein the biological sample was obtained from a subject that has or had a Clostridioides difficile infection (CDI) and is currently being treated with antibiotics, (b) contacting a substrate to which TcdB is attached with (i) the biological sample and (ii) a labeled antibody that binds to TcdB, (c) washing the substrate, (d) detecting a signal from the labeled antibody, wherein the amount of the signal is inversely correlated to the amount of neutralizing antibodies that bind TcdB in the sample, and (e) halting treatment of the subject with antibiotics if the amount of the signal is above a predetermined threshold.

In some embodiments, the subject has or had a primary CDI infection. In some embodiments, the subject has or had recurrent CDI infections.

In some embodiments, the predetermined threshold is determined by an aggregate of clinical samples. For example, the aggregate of clinical samples may comprise about 50 clinical samples, about 100 clinical samples, about 200 clinical, about 300 clinical, about 300 clinical, about 400 clinical, about 500 clinical, about 600 clinical, about 700 clinical, about 800 clinical samples, about 900 clinical, or about 1000 or more clinical samples.

In some embodiments, the labeled antibody is a neutralizing anti-TcdB antibody. For example, the labeled antibody may be selected from ZINPLAVA™ (bezlotoxumab)) N2-IgG, N3-IgG, N11-IgG, 2D-IgG, 2Ds-IgG, 5D-IgG, E3-IgG, 7F-IgG, B7-IgG, B12-IgG, or BB-IgG.

In some embodiments, the antibody that binds to TcdB in the sample is a neutralizing anti-TcdB antibody.

In some embodiments, the biological sample is selected from whole blood, isolated blood cells, plasma, serum, feces, or urine.

In some embodiments, the labeled antibody comprises a label selected from a tag, a fluorophore, an enzyme, a gold particle, a magnetic particle, a dye, or a radiolabel/isotope. In some embodiments, the tag can be biotin or epitope tag.

In some embodiments, the signal is selected from fluorescence, electrochemical, chemiluminescence, or bioluminescence.

In some embodiments of the therapeutic methods, the therapeutic is an antibody treatment. For example, the antibody treatment can be selected from FZ003 or (ZINPLAVA™ (bezlotoxumab)). In some embodiments, the treatment is delivered to the subject by intraperitoneal administration, intramuscular administration, intravenous administration, intrathecal administration, intranasal administration, or oral administration.

In another aspect, the present disclosure provides kits comprising:
(a) a Clostridioides difficile (C. diff) toxin B (TcdB)-coated substrate; and
(b) a labeled antibody that binds to TcdB.

In some embodiments, the kits can further comprise an unlabeled antibody that binds to TcdB. In some embodiments, the unlabeled antibody is the same or different from the labeled antibody that binds to TcdB.

In some embodiments, the labeled antibody comprises a label selected from a tag, a fluorophore, an enzyme, a gold particle, a magnetic particle, a dye, or a radiolabel/isotope. In some embodiments, the tag is biotin or epitope tag and the kit may further comprise a streptavidin-labeled signaling molecule. In some embodiments, the streptavidin-labeled signaling molecule is an enzyme, such as horseradish peroxidase. For the purposes of the disclosed kits, the label may be attached to the antibody directly (e.g., via a peptide bond or a chemical linker) or indirectly (e.g., via biotin/streptavidin).

In some embodiments, the labeled antibody is a neutralizing anti-TcdB antibody. For example, the antibody may be selected from ZINPLAVA™ (bezlotoxumab) N2-IgG, N3-IgG, N11-IgG, 2D-IgG, 2Ds-IgG, 5D-IgG, E3-IgG, 7F-IgG, B7-IgG, B12-IgG, or BB-IgG.

In another aspect, the present disclosure provides a kit comprising a TcdB-coated plate, an enzyme-labeled BB-IgG, and a chemical substrate for the enzyme, wherein the labeled BB-IgG comprises horseradish peroxidase (HRP) conjugated directly or indirectly to BB-IgG. In some embodiments, the kit may further comprise unlabeled BB-IgG

The foregoing general description and following detailed description are exemplary and explanatory and are intended to provide further explanation of the disclosure as claimed. Other objects, advantages, and novel features will be readily apparent to those skilled in the art from the following brief description of the drawings and detailed description of the disclosure.

### BRIEF DESCRIPTION OF THE DRAWINGS

FIG. 1 shows the structure of TcdB. The structure of a CROPs-deleted TcdB (PDB code 4R04) is used as an example. The CROPs is composed of four repeats.

FIG. 2 shows mice survival data after intraperitoneal injection (IP) with PBS, E3, a neutralizing VHH (10 μg/mouse), or a mixture of non-neutralizing V_{H}Hs (C6, C12, B7, and B12, 10 μg of each/mouse) 1 hour prior to ip challenge with 200 ng/mouse of TcdB. Mouse survival was monitored.

FIG. 3 shows the effects of CDI patient protective sera on toxins' cytotoxicity. 5× serially diluted recombinant TcdA or TcdB were mixed in the sera or PBS for 1 hr, and the samples were then applied to cells overnight. Cell rounding was quantified under a phase contrast microscope.

FIG. 4 shows a comparison of the level of neutralizing and the association with severe CDI. Panel A demonstrates a comparison of anti-toxin antibodies in CDI patients (light grey box and healthy donors (white box). Panels B and C demonstrate the levels of toxin-specific neutralizing antibodies against TcdA (Panel B) and TcdB (Panel C) and their associated with disease severity of CDI evaluated by SHEA/IDSA guideline. Patients with mild-to-moderate disease are represented by M (cross bar); patients with severe disease are represented by S (grey bar); patients with severe complicated CDI are represented with SC (dot bar). Antibody level is expressed as log2 transformed titer. Unpaired t test analysis was performed to compare the antibody levels between these two groups.

FIG. 5 shows analysis of three mAbs against TcdB cloned from B cell of donor #35. Amino acid sequences of the V region in the human monoclonal anti-TcdB antibodies (Panel A). FIG. 5A discloses SEQ ID NOS 21-24, 23, 25-27, and 23-24, respectively, in order of appearance. ELISA OD reading of the mAbs in binding with full-length of TcdB and the labeled fragments. ABAB-IgG is a chimeric antibody against GTD of TcdB serving as a control (Panel B). Competition ELISA of the listed antibodies (N2, N3, N4, N11, and ZINPLAVA™ (bezlotoxumab)) with ZINPLAVA™ (bezlotoxumab)). (Panel C). Neutralizing activities of the mAbs against TcdB-induced cell rounding. N3 showed the highest neutralizing activities. Merck anti-TcdA (Actoxumab) antibody was used as a negative control (Panel D).

FIG. 6 shows the percentage of inhibition of neutralizing anti-TcdB titers in competitive ELISA assay. Approximately 30 serum samples were grouped into high (>=1280) (Panel A), medium (between 80-640) (Panel B), and low (<=40 or undetectable) (Panel C) neutralizing anti-TcdB titers as determined by cell-based neutralizing cytotoxin assay. The 1^{st} open bar represents N2, the 2^{nd} light grey bar represents N3, the 3^{rd} dark grey bar represents N11, and the 4^{th} black bar represents BB-IgG1. The data showed that the levels of neutralizing anti-TcdB titers in CDI patients' samples were associated with percentage of inhibition of BB-IgG1 binding to TcdB.

FIG. 7 shows BB-IgG-based competitive ELISA to measure neutralizing antibodies in serum samples from 147 CDI patients. Panel A shows the correlation analysis that BB-ELISA results of the serum samples were significantly correlated to their neutralizing titers; Panel B shows the BB-ELISA results grouped according to their neutralizing titers determined by cell-based neutralization assay.

FIG. 8 shows a schematic of the C. Diff. NAb Detect™ IgG competitive ELISA kit (BB-ELISA Kit).

### DETAILED DESCRIPTION

Described herein, are methods of detecting neutralizing antibodies that bind to Clostridioides difficile (C. diff) toxin B (TcdB), prognosing the disease severity of Clostridioides difficile infection (CDI), determining the risk of primary and recurrence of CDI, and providing a guide for clinical practice for treating CDI.

### I. Definitions

Embodiments according to the present disclosure will be described more fully hereinafter. Aspects of the disclosure may, however, be embodied in different forms and should not be construed as limited to the embodiments set forth herein. Rather, these embodiments are provided so that this disclosure will be thorough and complete, and will fully convey the scope of the disclosure to those skilled in the art. The terminology used in the description herein is for the purpose of describing particular embodiments only and is not intended to be limiting.

Unless otherwise defined, all terms (including technical and scientific terms) used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the present application and relevant art and should not be interpreted in an idealized or overly formal sense unless expressly so defined herein. While not explicitly defined below, such terms should be interpreted according to their common meaning.

The practice of the present technology will employ, unless otherwise indicated, conventional techniques of tissue culture, organic chemistry pharmacology, immunology, molecular biology, microbiology, and cell biology, which are within the skill of the art. See, e.g. Current Protocols In Molecular Biology (F. M. Ausubel, et al. eds., (1987)); the series Methods in Enzymology (Academic Press, Inc.); Harlow and Lane, eds. (1988) Antibodies, a Laboratory Manual, and Animal Cell Culture (R. I. Freshney, ed. (1987)).

Unless the context indicates otherwise, it is specifically intended that the various features of the disclosure described herein can be used in any combination. Moreover, the disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

All numerical designations, e.g., pH, temperature, time, concentration, and molecular weight, including ranges, are approximations which are varied (+) or (−) by increments of 1.0 or as appropriate, or alternatively by a variation of +/−15%, or alternatively 10%, or alternatively 5%, or alternatively 2%. It is to be understood, although not always explicitly stated, that all numerical designations are preceded by the term “about”. It also is to be understood, although not always explicitly stated, that the reagents described herein are merely exemplary and that equivalents of such are known in the art.

As used in the description of the disclosure and the appended claims, the singular forms “a,” “an” and “the” are intended to include the plural forms as well, unless the context clearly indicates otherwise

As used herein, the term “about” refers to a measurable value such as an amount or concentration and the like, is meant to encompass variations of 20%, 10%, 5%, 1%, 0.5%, or even 0.1% of the specified amount

As used herein, the term “comprising” is intended to mean that the compositions and methods include the recited elements, but not excluding others. As used herein, the term “consisting essentially of” when used to define compositions and methods, shall mean excluding other elements of any essential significance to the combination. For example, a composition or method consisting essentially of the elements as defined herein would not exclude other elements that do not materially affect the basic and novel characteristic(s) of the claimed invention. As used herein, “consisting of” shall mean excluding more than trace amounts of other ingredients and substantial method steps recited. Embodiments defined by each of these transition terms are within the scope of this disclosure.

As used herein, the terms “acceptable,” “effective,” or “sufficient” refer to the selection of any components, ranges, dose forms, etc. disclosed herein intend that said component, range, dose form, etc. is suitable for the disclosed purpose.

As used herein, “and/or” refers to and encompasses any and all possible combinations of one or more of the associated listed items, as well as the lack of combinations when interpreted in the alternative (“or”).

As used herein, “Clostridioides difficile (C. diff) toxin B (TcdB)” refers to a cytotoxin produced by the bacterial Clostridioides difficile that has a molecular weight of 270 kDa and four different structural domains to include catalytic, cysteine protease, translocation, and receptor binding.

As used herein, “Clostridioides difficile infection (CDI)” or “Clostridium difficile colitis” or “pseudomembranous colitis” refers to an inflammation of the colon caused by the bacteria Clostridium difficile.

As used herein, “recurrent CDT” or “recurrence” refers to CDI that has occurred in a subject more than once.

As used herein, “prognosing” refers to determining the likelihood of current or future infections of a disease or the severity of the disease. In one embodiment, prognosing refers to predicting primary CDI. In one embodiment, refers to predicting the likelihood of recurrent CDI. In one embodiment prognosing refers to predicting disease severity.

As used herein, “primary CDI” refers to the first CDI infection in a subject.

As used here, “individual,” “subject,” and “patient” are used interchangeably herein, and refer to any individual mammalian, reptile, or bird subject, e.g., bovine, canine, feline, equine, porcine, poultry, or human. In some embodiments, the subject is a human.

As used herein, “treat,” “treatment,” or “treating” refers to reducing, ameliorating, or eliminating CDI.

As used herein, “prevent,” “preventing” or “prevention” refers to precluding or reducing the risk of developing CDI.

As used herein, “biological sample” refers to gathered matter of a subject's tissue, fluid, or other material derived from the subject. Non-limiting examples of biological samples include blood, serum, and plasma, as well as tissue samples and other bodily fluids.

As used herein, the term “antibody” collectively refers to immunoglobulins or immunoglobulin-like molecules including IgA, IgD, IgE, IgG and IgM, combinations thereof or fragments thereof. Fragments of antibodies may include, for example, Fab fragments and single chain variable fragments (scFv). An antibody generally comprises heavy (H) chains and light (L) chains interconnected by disulfide bonds. There are two types of light chain, lambda (λ) and kappa (κ). The term “antibody” additionally includes single-domain antibodies (i.e., a V_{H}H antibody or a “camelid-like” antibody). The term “antibody” is further intended to encompass digestion fragments, specified portions, derivatives and variants thereof, including antibody mimetics or comprising portions of antibodies that mimic the structure and/or function of an antibody or specified fragment or portion thereof, including single chain antibodies and fragments thereof. There are five main heavy chain classes (or isotypes) which determine the functional activity of an antibody molecule: IgM, IgD, IgG, IgA and IgE. Each heavy and light chain contains a constant region and a variable region (also known as “domains”). In combination, the heavy and the light chain variable regions, also called the “Fab region,” specifically bind to a given antigen. Light and heavy chain variable regions contain a “framework” region interrupted by three hypervariable regions, also called “complementarity-determining regions” or “CDRs.” The extent of the framework region and CDRs has been defined (see Kabat et al., Sequences of Proteins of Immunological Interest, U.S. Department of Health and Human Services, 1991). The Kabat database is now maintained online. The sequences of the framework regions of different light or heavy chains are relatively conserved within a species, and framework regions act to form a scaffold that provides for positioning the CDRs in correct orientation by inter-chain, non-covalent interactions.

The CDRs are primarily responsible for binding to an epitope on an antigen. The CDRs of each chain are typically referred to as CDR1, CDR2, and CDR3, numbered sequentially starting from the N-terminus, and are also typically identified by the chain in which the particular CDR is located. Thus, a HCDR3 is located in the variable domain of the heavy chain of the antibody in which it is found, whereas a LCDR1 is the CDR1 from the variable domain of the light chain of the antibody in which it is found. An antibody that binds IL-31RA will have a specific V_{H} region and the V_{L} region sequence, and thus specific CDR sequences. Antibodies with different specificities generally have different CDRs. Although it is the CDRs that vary from antibody to antibody, only a limited number of amino acid positions within the CDRs are directly involved in antigen binding. These positions within the CDRs are called specificity determining residues (SDRs).

The Fc fragment region (Fc) of an antibody plays a role in modulating immune cell activity. The Fc region functions to guarantee that each antibody generates an appropriate immune response for a given antigen, by binding to a specific class of proteins found on certain cells, such as B lymphocytes, follicular dendritic cells, natural killer cells, macrophages, neutrophils, etc. and are called “Fc receptors.” Because the constant domains of the heavy chains make up the Fc region of an antibody, the classes of heavy chain in antibodies determine their class effects. The heavy chains in antibodies include alpha, gamma, delta, epsilon, and mu, and correlate to the antibody's isotypes IgA, IgG, IgD, IgE, and IgM, respectively. Thus, different isotypes of antibodies have different class effects due to their different Fc regions binding and activating different types of receptors.

There are four subclasses of IgG, which is the most abundant antibody isotype found in human serum. The four subclasses, IgG1, IgG2, IgG3, and IgG4, which are highly conserved. The amino acid sequence of the constant regions of these peptides are known in the art, e.g., see Rutishauser, U. et al. (1968) “Amino acid sequence of the Fc region of a human gamma G-immunoglobulin” PNAS 61(4):1414-1421; Shinoda et al. (1981) “Complete amino acid sequence of the Fc region of a human delta chain” PNAS 78(2):785-789; and Robinson et al. (1980) “Complete amino acid sequence of a mouse immunoglobulin alpha chain (MOPC 511)” PNAS 77(8):4909-4913.

As used herein, “neutralizing antibody (Nab)” refers to an antibody that defends a cell from a toxin, pathogen or infectious particle by neutralizing the biological effects of the toxin, pathogen or infectious particle.

### II. C. diff Infection (CDI), TcdA and TcdB

Symptoms associated with C. diff. infection (CDI) are mainly caused by two major exotoxins, TcdA and TcdB. TcdB is the key virulence factor of C. diff. CDI recurrence is difficult to treat and causes lengthy hospitalization and significant financial losses. Current standard treatment for CDI is antibiotics. A non-intended side effect of antibiotic is disruption of the microbiota colonic flora, allowing C. diff to flourish, leading to the elaboration of toxin A and toxin B, which causes mucosal inflammation and injury. The CDI recurrence rate approaches 35%. The risk of further episodes of CDI in recurrent patients can be more than 50% and a subset of patients will have multiple recurrences. Recurrent CDI can be caused by the same strain or newly colonizing strains. Mild CDI can manifest as watery diarrhea (up to 10-15 times a day), abdominal pain, cramping, fever, and leukocytosis. Symptoms can progress in moderate to severe cases with the development of sepsis, pseudomembranous colitis or fulminant colitis with bowel perforation, toxic megacolon, and death. See Cole, S. A., & Stahl, T. J. (2015). Persistent and Recurrent Clostridium difficile Colitis. Clinics in colon and rectal surgery, 28(2), 65-69. It is a frustrating condition that is difficult to manage and may affect patients for months or even years, causing tremendous morbidity and mortality. The high rate of recurrence is the most significant issue in clinical management of CDI.

In response to C. diff. infection, the immune system should make toxin-neutralizing antibodies (antitoxin NAbs) against TcdA and TcdB. Nabs are responsible for effective immunity and prevention of CDI. Knowing the protective immune response level in patients with C. Diff. infection is crucial for optimal clinical management and improved prognosis. Being able to determine whether a person is infected with C. Diff and has mounted enough antitoxin NAbs so that they are unlikely to develop severe CDI or unlikely to suffer recurrence would inform selection of the optimal treatment regime, greatly improve patient outcomes and reduce CDI recurrence. Conversely, patients with a robust anti-toxin immune response may need fewer or shorter duration treatments thus reducing use of unnecessary antibiotics, which reduces the likely hood of strains of C. diff. to become more virulent resistant “super bugs”.

Patients that develop strong protective immune responses to C. diff. toxins are less likely to have a relapse. However, there are no commercially available tests for assessing the strength of a patient's protective immune response to C. diff. toxins. A test that can correlate the amount of patient neutralizing antibodies against C. diff. toxins to the likelihood of CDI recurrence could be used to predict recurrence risk. If a patient is vulnerable to recurrence, then the optimal therapeutic strategy might include FDA approved anti-toxins (e.g., ZINPLAVA™ (bezlotoxumab)) in addition to standard of care antibiotics (fidaxomicin, or metronidazole, or vancomycin) for the prevention of recurrent CDI. Conversely, patients with a robust anti-toxin immune response may need fewer or shorter duration treatments.

Several factors contribute to disease severity including protective anti-toxin levels with severity being associated with low anti-toxins. Therapeutic antibody treatment requires quick action to block disease progression. Considering that antibody therapy is expensive, there is currently a clinical need to measure neutralizing anti-toxin levels.

There is no commercially available test to measure patient antitoxin NAbs in response to CDI. Current cell culture based non-commercial assays take advantage of serum inhibition of toxins' biological activities on cultured cells. However, such assays are time consuming, laborious, require specialized equipment, and are not standardized. The detection process utilizing cultured cells takes about 40 hrs. In addition, the cells need to be maintained twice a week. As low as 10 pg/ml of toxin B used in this assay can cause 100% cell rounding in the absence of toxin-neutralizing antibody block. This assay relies on toxins' biological activity, which is very sensitive to the freeze/thaw damage. In addition, the toxins' biological activity is compromised when diluted to the low concentration level required for the cultured cell assay. In addition, to reduce the errors between experiments, commercial human serum is used for quality control for the cultured cell detection system. To determine the titer of a patient serum sample, each sample requires performing duplicates of up to 8 continuous 2× serial dilutions. Therefore, this cell culture assay requires a large patient sample size for testing and also requires replicates. Thus, these cell culture bioassays are limited to research laboratories and have not had any significant clinical impact and there is a significant technological problem to be solved in this regard.

### CDI Recurrence

Current standard treatment for CDI with antibiotics causes the disruption of the microbiota and results in a relapse rate approaching 35%. The risk of further episodes of CDI in recurrent patients can be more than 50% and a subset of patients will have multiple recurrences. Recurrent CDI can be caused by the same strain or newly colonizing strains. It is a frustrating condition that is difficult to treat and may affect patients for months or even years, causing tremendous morbidity and mortality.

### TcdA and TcdB Play Essential Roles in the Pathogenesis of CDI

CDI is mainly caused by the two exotoxins TcdA and TcdB, because TcdA⁻TcdB⁻ strains are avirulent. The two toxins are structurally similar and exhibit a similar mode of action on host cells. Both toxins consist of four functional domains: the N-terminal glucosyltransferase domain (GTD), a cysteine protease domain (CPD) that mediates autocleavage and releases GTD into host cytosol, a central hydrophobic region (TD) that may be involved in transmembrane delivery of GTD, and a C-terminal receptor-binding domain (RBD; also known as combined repetitive oligopeptides, or CROPs) involved in receptor binding (FIG. 1). Both toxins target host Rho GTPases, leading to their inactivation as well as cytoskeleton disorganization, which is the cause of cell morphology changing into round shape. The relative roles of the two toxins in the pathogenesis of CDI are not well understood, but recent studies have shown that TcdB is the most important virulence factor in animals. All pathogenic C. diff. isolates from human secrete TcdB. Some strains of C. diff. produce a third toxin, called binary toxin (CDT), but it is not essential for the pathogenesis of the bacteria since TcdA⁻TcdB⁻CDT⁺ strains are avirulent.

All Pathogenic C. diff. Produces TcdB and Host Anti-TcdB Nabs Confer Protection Against the Infection. Furthermore, Neutralizing Antibodies Against TcdB Protect Against CDI, and are Associated With Disease Severity in Patients.

In a recent large multi-center, controlled clinical study of CDI patients, toxin-specific neutralizing titers were found to be associated with disease severity in patients while total toxin-binding activities were not. Reduced levels of neutralizing antibodies against TcdB, but not TcdA, were found in patients with more severe disease compared to those with mild-to-moderate disease according to Infectious Disease Society of America (IDSA)/Society of Healthcare Epidemiology of America (SHEA) guidelines. These animal and clinical studies further prove a crucial role of neutralizing antibodies against TcdB in protection against CDI disease.

Numerous independent studies have demonstrated that systemic and mucosal antibodies against the toxins confer protection against CDI. Recently, a report showed that an anti-TcdB, but not anti-TcdA, neutralizing antibody (ZINPLAVA™ (bezlotoxumab)) conferred protection against CDI in gnotobiotic piglets. ZINPLAVA™ has been validated in a phase III clinical trial and subsequently approved by FDA as a prevention against recurrent.

### Only Anti-TcdA and Anti-TcdB Antibodies With Neutralizing Activities are Protective in Animals.

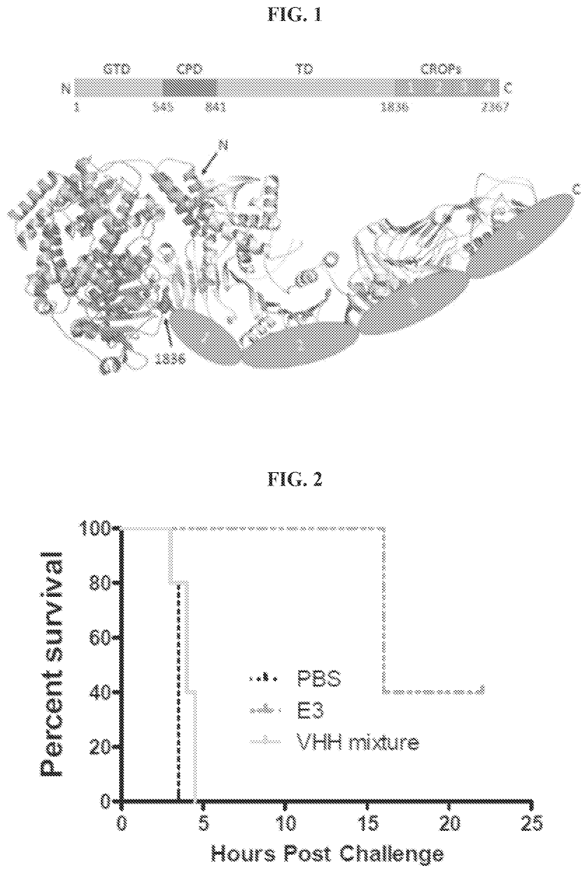

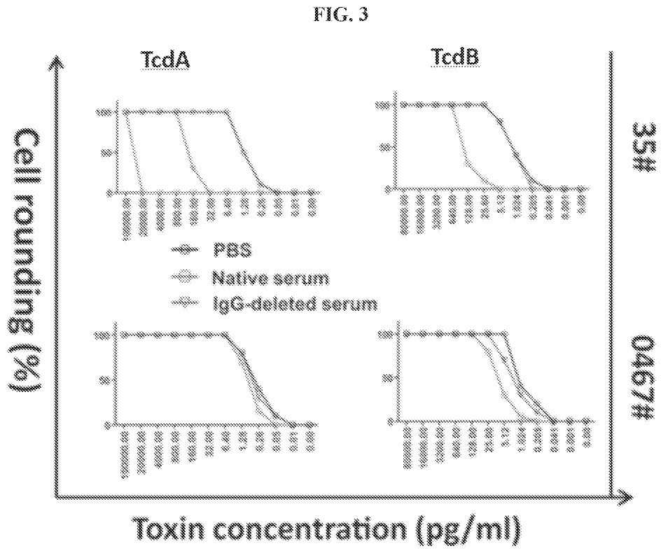

Some antibodies specific to C. diff. toxins enhance the toxins' activities, i.e., cytotoxicity and inflammatory activity, and thus may be detrimental to protection against CDI; some antitoxin antibodies have no effect on the toxins' activities; and some antibodies that neutralize the two toxins are highly protective against CDI in animal models. Neutralizing monoclonal V_{H}H antibody E3 is highly protective, whereas a mixture of high affinity, toxin-specific, non-neutralizing V_{H}Hs have no protective effect against lethal systemic TcdB challenge in mice (FIG. 2). The fact that single domain E3 antibody (devoid of Fc) is protective in mice suggests that the protection against systemic TcdB challenge does not need Fc-mediated function. Further, Fc-mediated effector function is not necessary for antibody protection against CDI in mice. Finally, mouse CDI and ileal loop models were used to demonstrate that the mechanism of antibody-mediated protection is through toxin neutralization. All these in vitro and in vivo animal studies indicate that only neutralizing antibodies against the two toxins, but not non-neutralizing antibodies, block the toxins' activity and protect animals from CDI.

It is entirely novel to establish an ELISA-based assay for detecting neutralizing antitoxin response in CDI patients since such an assay is not available and never reported. It is also entirely novel to use a simple and quick serological ELISA assay to predict disease progress and recurrence in CDI patients. These novelties are built upon some of the findings in the following descriptions and/or Examples.

### Neutralizing Antibodies From Patients Block Cytotoxicity of TcdA and TcdB.

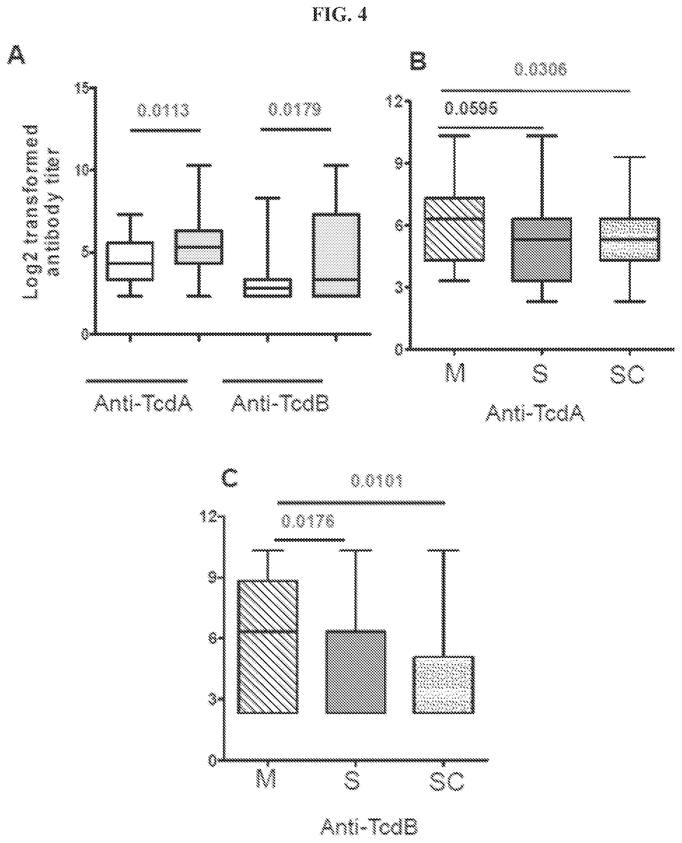

In a clinical study, the ability of anti-TcdA and anti-TcdB antibodies from CDI patient sera to block the toxins' cytotoxicity was assessed. The serum from patient #35, who has relatively high neutralizing titers against TcdB, significantly blocked TcdB cytotoxicity, whereas the serum from patient #0467 who has low or undetectable neutralizing titers, has no blocking effects, (FIG. 3). Consistent with these results, depleting serum of IgG only affects the toxin blocking activity of serum from patient #35 but not from patient #0467 (FIG. 3). This data demonstrated that neutralizing antibodies against the two toxins in patient sera play a critical role in blocking toxins' toxicity.

This disclosure relates to the development of a simple in vitro serological competition ELISA to measure a (CDI patient's NAb response to C. diff. toxins and predict disease recurrence and guide treatment options. The ELISA is designed for simplicity in manufacturing and performance, with binding attributes in clinically relevant ranges for CDI. The ELISA is standardized and routine with features that are easily adopted to automated methods, and easy integration into clinical practice for rapid diagnosis of protective immunity in CDI patients, which will aid clinicians in optimal disease management. This is the first commercially and clinically viable assay developed for detecting NAb response in CDI patients. It is also the first assay developed to predict disease progression and recurrence in CDI patients. This assay will be a valued and impactful addition to the clinical tools available for the management of CDI and greatly improve patient outcomes. This is an unconventional technical solution that solves the technical problem in the field.

### Neutralizing Anti-TcdB Antibodies in Patients are Associated With Disease Severity.

Greater than 60% of healthy children, aged greater than 2 years, and adults have detectable serum IgG and IgA antibodies to C. diff. TcdA and TcdB, even in the absence of C. diff. colonization or active infection. Since antibody-mediated protection is through toxin neutralization (Section B1 and B2), determining the correlation of neutralizing antitoxins with disease severity in CDI patients would be useful. In a previous study, approximately 100 banked serum samples from healthy donors (30) and CDI patients (˜70) were analyzed. In response to CDI, increased levels of toxin-specific neutralizing antibodies were induced in patients (FIG. 4A). Significantly, the reduced levels of neutralizing antibodies against TcdB toxins were found in patients with more severe disease compared to those with mild-to-moderate disease according to SHEA\IDSA guidelines (FIG. 4C). A similar tendency was seen in the association of neutralizing anti-TcdA level and CDI severity, but was not significant (p=0.0595) (FIG. 4B). The levels of neutralizing anti-TcdA were compared between patients with mild-to-moderate disease and severe complicated CDI since this population has poor prognosis and often associated with death outcomes. As shown in FIG. 4B-C, both neutralizing anti-TcdA and anti-TcdB titers were significantly lower in severe complicated CDI patients (p=0.0306 for anti-TcdA and 0.0101 for anti-TcdB). The results indicated that neutralizing antitoxins, especially anti-TcdB, may play a key role in protecting CDI patients from progression into more severe diseases and recurrent CDI.

Identifying Neutralizing Antibodies Against C. diff. Toxins

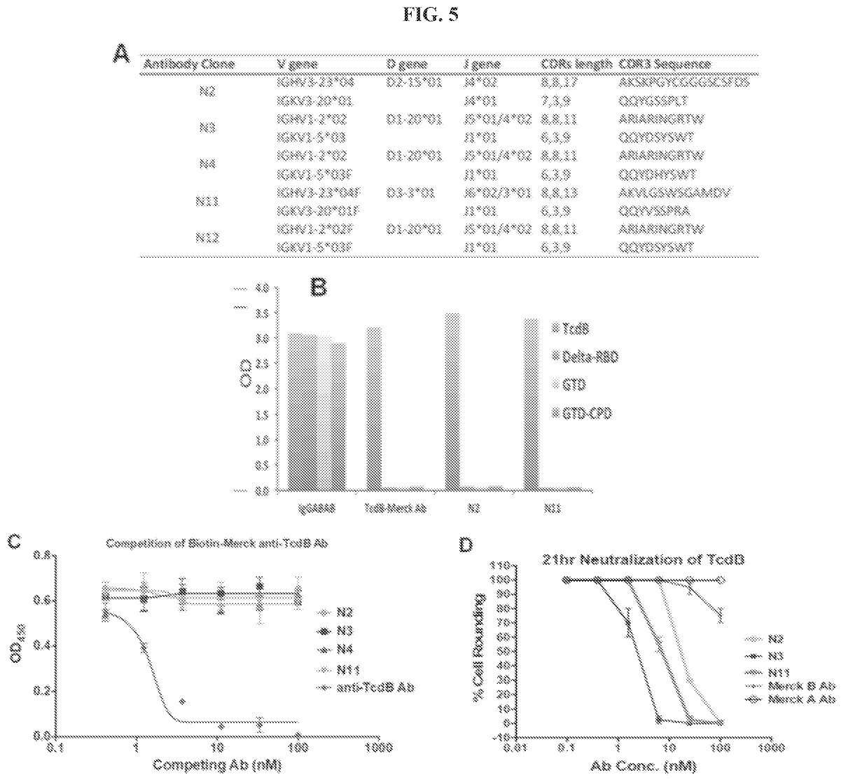

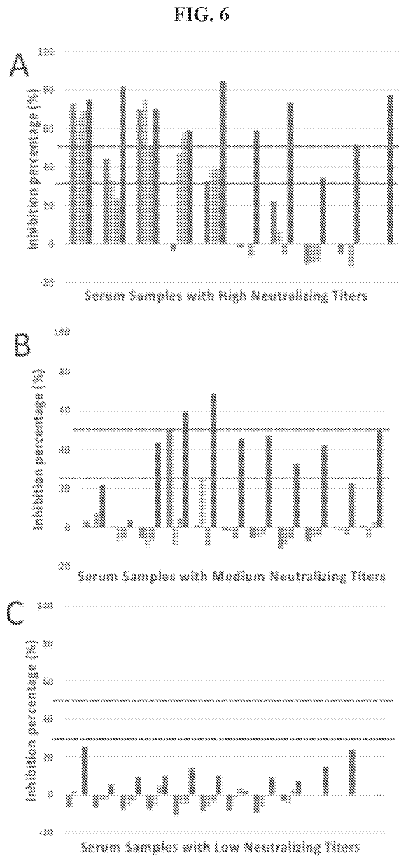

Using phage-display technology panels of neutralizing anti-TcdA and anti-TcdB monoclonal antibodies were identified. Moreover, using a high efficiency human mAb cloning technology to directly clone TcdA- and TcdB-specific antibody genes from the B cells from convalescent CDI patients, a panel of anti-TcdB human monoclonal antibodies (FIGS. 5A-5D) was generated. Five pairs of TcdB-specific antibody heavy and light chain genes were obtained, of which there are 3 unique mAbs, N2, N3 and N11; N4 and N12 are clonal mAbs of N3 (FIG. 5A). The higher frequency of N3 than other antibodies suggests that N3 is an immunodominant antibody. N2 and N11 were found to bind to CROPs of TcdB (FIG. 5B). N3 may be targeting a conformational epitope that is associated with the CROPs of TcdB (FIG. 5B). It has been previously shown, through crystal structure analysis of N3-TcdB complex that N3 indeed binds to C-terminus of TD adjacent CROPs of TcdB. As for neutralizing activity, N3 has the highest potency followed by N11, N2 and the ZINPLAVA™ (bezlotoxumab)) (FIG. 5D). These neutralizing antibodies allow us to select those immunodominant antibodies for serological ELISA assay as described herewith.

### III. Methods for Detecting Antibodies That Bind to C. diff. Toxin B (TcdB)

Embodiments described herein generally relate to methods of detecting antibodies that bind to C. diff. toxin (TcdB). The methods can lead to prognosis or determining recurrence of Clostridioides difficile infection (CDI). The disclosed methods can be semi-quantitative, and rely on the intensity of a marker signal to establish the amount of anti-TcdB antibodies in a biological sample (e.g., blood, serum, or plasma) from a patient with or suspected of having CDI (either primary or recurrent).

Disclosed herein are methods of detecting antibodies that bind to Clostridioides difficile (C. diff) toxin B (TcdB) in a biological sample, comprising: (a) contacting a substrate to which TcdB is attached with (i) a biological sample and (ii) a labeled antibody that binds to TcdB, (b) washing the substrate, and (c) detecting a signal from the labeled antibody, wherein the amount of the signal detected is inversely correlated to the amount of antibodies that binds to TcdB in the sample; wherein the biological sample was obtained from a subject that had a Clostridioides difficile infection (CDI) prior to obtaining the sample.

Disclosed herein are methods of prognosing Clostridioides difficile infection (CDI), comprising: (a) contacting a substrate to which Clostridioides difficile (C. diff) toxin B (TcdB) is attached with (i) a biological sample and (ii) a labeled antibody that binds to TcdB, (b) washing the substrate, and (c) detecting a signal from the labeled antibody, wherein a signal intensity that is below a predetermined threshold is indicative of a primary CDI or a risk of recurrent CDI, and a signal intensity that is above the predetermined threshold is indicative of lack of primary CDI or a low risk of recurrent CDI; wherein the biological sample was obtained from a subject that had or has a Clostridioides difficile infection (CDI).

Disclosed herein are methods of determining recurrence of Clostridioides difficile infection (CDI), comprising: (a) contacting a substrate to which Clostridioides difficile (C. diff) toxin B (TcdB) is attached with (i) a biological sample and (ii) a labeled antibody that binds to TcdB, (b) washing the substrate, and (c) detecting a signal from the labeled antibody, wherein a signal intensity that is below a predetermined threshold is indicative of recurrent CDI or a risk thereof; wherein the biological sample was obtained from a subject that had or has a Clostridioides difficile infection (CDI).

Disclosed herein are methods of treating Clostridioides difficile infection (CDI) in a subject, comprising: (a) obtaining a biological sample, wherein the biological sample was obtained from a subject that had a Clostridioides difficile infection (CDI) prior to obtaining the sample (b) contacting a substrate to which TcdB is attached with (i) the biological sample and (ii) a labeled antibody that binds to TcdB, (c) washing the substrate, (d) detecting a signal from the labeled antibody, wherein the amount of the signal is inversely correlated to primary CDI or development recurrent CDI, and (e) treating the subject with a therapeutic for CDI if the amount of the signal is below a predetermined threshold.

In some embodiments, the amount of signal can be correlated against a predetermined threshold. The predetermined threshold can be determined by an aggregate of clinical samples. In some embodiments, the aggregate of clinical samples comprises about 50 clinical samples, about 100 clinical samples, about 200 clinical, about 300 clinical, about 400 clinical, about 500 clinical, about 600 clinical, about 700 clinical, about 800 clinical samples, about 900 clinical, or about 1000 or more clinical samples. In some embodiments, the aggregate of clinical samples comprises at least 50 clinical samples, at least 100 clinical samples, at least 200 clinical, at least 300 clinical, at least 400 clinical, at least 500 clinical, at least 600 clinical, at least 700 clinical, at least 800 clinical samples, at least 900 clinical, or at least 1000 or more clinical samples. In some embodiments, the clinical samples comprises between about 50 to about 100 clinical samples, between about 50 to about 150 clinical samples, between about 50 to about 200 clinical samples, between about 100 to about 150 clinical samples, between about 100 to about 200 clinical samples, between about 100 to about 250 clinical samples, between about 150 to about 200 clinical samples, between about 150 to about 250 clinical samples, between about 150 to about 300 clinical samples, between about 200 to about 250 clinical samples, between about 200 to about 300 clinical samples, between about 200 to about 350 clinical samples, about 250 to about 300 clinical samples, between about 250 to about 350 clinical samples, between about 250 to about 400 clinical samples, about 300 to about 350 clinical samples, between about 300 to about 400 clinical samples, between about 300 to about 450 clinical samples, about 350 to about 400 clinical samples, between about 350 to about 450 clinical samples, between about 350 to about 500 clinical samples, about 400 to about 450 clinical samples, between about 400 to about 500 clinical samples, between about 400 to about 550 clinical samples, about 450 to about 500 clinical samples, between about 450 to about 550 clinical samples, between about 450 to about 600 clinical samples, about 500 to about 550 clinical samples, between about 500 to about 600 clinical samples, between about 500 to about 650 clinical samples, about 550 to about 600 clinical samples, between about 550 to about 650 clinical samples, between about 550 to about 700 clinical samples, about 600 to about 650 clinical samples, between about 600 to about 700 clinical samples, between about 600 to about 750 clinical samples, about 750 to about 800 clinical samples, between about 750 to about 850 clinical samples, between about 750 to about 900 clinical samples, about 800 to about 850 clinical samples, between about 800 to about 900 clinical samples, between about 800 to about 950 clinical samples, about 850 to about 900 clinical samples, between about 850 to about 950 clinical samples, between about 850 to about 1000 clinical samples, about 900 to about 950 clinical samples, between about 900 to about 1000 clinical samples, or between about 950 to about 1000 clinical samples.

In some embodiments, the labeled antibody is a neutralizing anti-TcdB antibody. In one embodiment, the labeled antibody is selected from ZINPLAVA™ (bezlotoxumab)) N2-IgG, N3-IgG, N11-IgG, 2D-IgG, 2Ds-IgG, 5D-IgG, E3-IgG, 7F-IgG, B7-IgG, B12-IgG, or BB-IgG. In one embodiment, the labeled antibody is selected from the group consisting of ZINPLAVA™ (bezlotoxumab)) N2-IgG, N3-IgG, N11-IgG, 2D-IgG, 2Ds-IgG, 5D-IgG, E3-IgG, 7F-IgG, B7-IgG, B12-IgG, or BB-IgG. In one embodiment, the labeled antibody is N2-IgG. In one embodiment, the labeled antibody is N3-IgG. In one embodiment, the labeled antibody is N11-IgG. In one embodiment, the labeled antibody is BB-IgG. In one embodiment, the antibody that binds to TcdB in the sample is a neutralizing anti-TcdB antibody. The antibody sequences of N2-IgG, N3-IgG, N11-IgG are found in NIH Grant Nos 1R43AI136176-01 and 2R44AI136176-02, which are incorporated by reference herein. The sequences of the CDRs (CDR1, CDR2, and CDR3) of the neutralizing antibodies are provided in Table 1. Additional CDR sequences are provided in FIG. 5A.

Each of the disclosed antibodies 2D, 2Ds, 5D, E3, and 7F were initially isolated as V_{H}H antibodies, which are single domain, heavy-chain only antibodies and do not include a light chain and CH domains. These antibodies were subsequently humanized, and both the humanized and original alpaca forms of the antibodies can be used in the disclosed kits and methods. While V_{H}H antibodies are suitable for use in the disclosed methods, in some embodiments it can be advantageous to reformate these V_{H}H antibodies as IgGs (e.g., an IgG1, IgG2, IgG3, or IgG4). In such a reformatting process, the V_{H}H can be fused with an IgG backbone by removing VL and VH of the IgG and replacing each of these variable domains with the V_{H}H. Thus, the resulting V_{H}H-IgG has four V_{H}H domains (one in place of each VH and VL domain) and the constant regions (CH1, CH2, CH3, and CL) and hinge region of an IgG. For example, 5D-IgG comprises one 5D in each of the light chain variable domain positions of an IgG and one 5D in each of the heavy chain variable domain positions of the IgG. BB is a bispecific antibody, which comprises a 5D domain and an E3 domain. BB-IgG is therefore a bispecific IgG, which comprises two 5D V_{H}Hs in place of the light or heavy chain variable domains and two E3 V_{H}Hs in place of the light or heavy chain variable domains, whichever is not replaced with 5D. Thus, in some embodiments, BB-IgG comprises two 5D V_{H}Hs, one each in place of the light chain variable domains of an IgG, and two E3 V_{H}Hs, on each in place of the heavy chain variable domains of the IgG. In some embodiments, BB-IgG comprises two 5D V_{H}Hs, one each in place of the heavy chain variable domains of an IgG, and two E3 V_{H}Hs, on each in place of the light chain variable domains of the IgG. For the purposed of the disclosed V_{H}H-IgGs, the IgG can be IgG1, IgG2, IgG3, or IgG4.

TABLE 1

| Antibody | CDR1 | CDR2 | CDR3 |
|---|---|---|---|
| 5D | GFTLDYYG | ISASARTI | ARRRFSAS |
| | (SEQ ID | (SEQ ID | SVNRWLA |
| | NO: 1) | NO: 2) | DDYDVW |
| | | | (SEQ ID |
| | | | NO: 3) |
| E3 | GSIAGFET | MTKTNNEI | KGPELR |
| | (SEQ ID | (SEQ ID | (SEQ ID |
| | NO: 4) | NO: 10) | NO: 15) |
| 2D | GFSLDYYG | ISASAKTK | ARRRFDA |
| | (SEQ ID | (SEQ ID | SASNRW |
| | NO: 5) | NO: 11) | (SEQ ID |
| | | | NO: 16) |
| 2Ds | SERNPER | WQTGGSLN | YLKKWRD |
| | NPG | (SEQ ID | QYW (SEQ |
| | (SEQ ID | NO: 12) | ID NO: |
| | NO: 6) | | 17) |
| 7F | GSSFSTST | FTSGGAI | ALHNAVS |
| | (SEQ ID | (SEQ ID | GSSW (SEQ |
| | NO: 7) | NO: 13) | ID NO: 18) |
| Bezlotoxumab | QSVSSSY | GAS | QQYGSS |
| VL | (SEQ | | TWTF |
| | ID NO: | | (SEQ ID |
| | 8) | | NO: |
| | | | 19) |
| Bezlotoxumab | GYSFTS | FYPGDSST | ARRRNW |
| VH | YW | (SEQ ID | GNAFDIW |
| | (SEQ ID | NO: 14) | (SEQ ID |
| | NO: 9) | | NO: 20) |

The antibodies used in the disclosed methods and kits can be “broadly neutralizing,” meaning that they bind to TcdB from various C. diff strains or isolates. In general, it is understood by those skilled in the art that any anti-TcdB antibody may be used for the purposes of the disclosed methods and kits, as the antibody need only to compete with antibodies in the biological sample (e.g., blood, serum, or plasma) in order to function for the disclosed purposes. However, the disclosed antibodies and BB, in particular, have been shown to broadly bind to TcdB from various strains, making them particularly well suited for the assays and kits described herein. That notwithstanding, other anti-TcdB antibodies known in the art may be suitable for incorporation into the disclosed methods and kits as well.

For the purposes of the disclosed methods, the substrate to which TcdB is attached is one that is suitable for enzyme-linked immunosorbent assays (ELISA) and similar assay formats. Such substrates are known in the art and include, but are not limited to, plates (e.g., microplates and strips) that comprise a base material of glass, plastic, polystyrene, or polycarbonate. Those skilled in the art will understand that a suitable substrate can be chosen based on properties, such as hydrophobicity, and that various commercially available alternative can be selected. For example a hydrophobic (e.g., PolySorp, Immulon 1 B, Microlite 1+, Microfluor 1) slightly hydrophilic (e.g., Immunlon 2 HB, Microlite 2+, Microfluor 2, MediSorp), hydrophilic (e.g., MaxiSorp, Immunlon 4 HBX), or very hydrophilic (e.g., MultiSorp) can be utilized.

The biological sample used for the disclosed methods is not particularly limited, so long as the biological sample is from the subject. In some embodiments, the biological sample is selected from whole blood, isolated blood cells, plasma, serum, feces or other biological fluids. In one embodiments, the biological sample is selected from the group consisting of whole blood, isolated blood cells, plasma, serum, feces and other biological fluids. In one embodiment, the biological sample is whole blood. In one embodiment, the biological sample is isolated blood cells. In one embodiment, the biological sample is plasma. In one embodiment, the biological sample is serum. In one embodiment, the biological sample is feces. In one embodiment, the biological sample is a bodily fluid.

The methods and kits disclosed herein rely on a labeled antibody to compete with the anti-TcdB antibodies in a biological sample from a patient to bind the TcdB fixed (i.e., attached or conjugated) to a substrate, such as a glass, plastic, polystyrene, or polycarbonate plate. The label is not particularly limited, so long as it produced a signal that can be correlated to the relative abundance of anti-TcdB antibodies in the biological sample competing with the labeled antibody. Commonly used labeled that are suitable for the disclosed methods include, but are not limited to protein tags (e.g., FLAG, MYC, etc.), a fluorophore (e.g., FITC, Texas Red, GFP, PE, etc.), enzymes (e.g., horseradish peroxidase or HRP, Alk Phosphatase), gold, magnetic particles, chemiluminescence agents, colorimetric regents, dyes, radiolabels/isotopes, and the like. When an enzyme is used as the label, it can be conjugated directly or indirectly to the labeled antibody. For example, a direct conjugation would be an anti-TcdB antibody with HRP (or other enzyme) directly attached via a peptide or chemical linker, whereas an indirect conjugation would be an anti-TcdB antibody conjugated to biotin (i.e., a biotinylated antibody) and a HRP (or other enzyme) conjugated to streptavidin.

Further, when a peroxidase, such as HRP is utilized as the label, the methods and kits will also include a chemical substrate (be it chromogenic, fluorescent, chemiluminescent, or colorimetric) on which the enzyme can act. Non-limiting examples include 3,3′,5,5′-Tetramethylbenzidine (TMB), hydroxypheny-lacetic acid, 3-p-hydroxyphenylproprionic acid (HPPA), luminol, polyphenols and acridine esters, and luciferin. Thus, in some embodiments in which an enzyme is used as the label, detecting the signal may comprise introducing a chemical substrate to the assay such that the enzyme acts on the chemical substrate to create a signal. Likewise, kits which utilize an enzyme as the label can optionally comprise a chemical substrate, such as 3,3′,5,5′-Tetramethylbenzidine (TMB), hydroxypheny-lacetic acid, 3-p-hydroxyphenylproprionic acid (HPPA), luminol, a polyphenol, an acridine ester, or luciferin.

In some embodiments, the labeled antibody comprises a label selected from a tag, a fluorophore, an enzyme, a gold particle, a magnetic particle, a dye, or a radiolabel/isotope. In one embodiment, the labeled antibody comprises a label selected from the group consisting of a tag, a fluorophore, an enzyme, a gold particle, a magnetic particle, a dye, and a radiolabel/isotope. In one embodiment, the label is a tag. In one embodiment, the label is a fluorophore. In one embodiment, the label is an enzyme. In one embodiment, the label is a gold particle. In one embodiment, the label is a magnetic particle. In one embodiment, the label is a dye. In one embodiment, the label is a radiolabel/isotope. In one embodiment, the tag is biotin. In one embodiment, the enzyme is a streptavidin-labeled signaling molecule. In one embodiment, the enzyme is horseradish peroxidase. In one embodiment, the enzyme is alkaline phosphatase. In one embodiment, the tag is an epitope tag. In one embodiment, the signal is selected from fluorescence, electrochemical, chemiluminescence, or bioluminescence

In some embodiments, an antibody can be monoclonal or polyclonal. Non-limiting examples of detecting a signal from a labeled antibody include Western blots, enzyme linked immunosorbent assays (ELISA), immunoprecipitations, and immunofluorescence.

In some embodiments, a subject is a mammal. In one embodiment, a subject is a human. In one embodiment, a subject had a primary CDI infection. In one embodiment, a subject has had recurrent CDI infection. In one embodiment, the subject has CDI. In one embodiment, the subject is male. In one embodiment, the subject is female. In one embodiment, the subject is a premature newborn. In one embodiment, a premature newborn is born before 36 weeks gestation. In one embodiment, the subject is a term newborn. In one embodiment, a term newborn is below about 2 months old. In one embodiment, the subject is a neonate. In one embodiment, a neonate is below 1 month old. In one embodiment, the subject is an infant. In one embodiment, an infant is between about 2 months to about 24 months old. In one embodiment, the subject is a toddler. In one embodiment, a toddler is between about 2 years old to about 4 years old. In one embodiment, the subject is a child. In one embodiment, a child is between about 5 years old and 12 years old. In one embodiment, the subject is an adolescent. In one embodiment, an adolescent is between about 13 years to about 19 years. In one embodiment, the subject is an adult. In one embodiment, an adult is between about 20 years to 95 or more years.

In some embodiments, the therapeutic is an antibody treatment. Non-limiting examples of therapeutic antibody treatment include FZ003 disclosed in International publication WO 2020/247500 A1 which is incorporated by reference herein. or (ZINPLAVA™ (bezlotoxumab)). In one embodiment, the therapeutic is delivered to the subject. In one embodiment, the treatment is delivered to the subject by intraperitoneal administration, intramuscular administration, intravenous administration, intrathecal administration, intranasal administration, or oral administration. In one embodiment, the therapeutic is delivered together with a pharmaceutically acceptable carrier. In one embodiment, a pharmaceutically acceptable carrier is a non-toxic solvent, dispersant, excipient, adjuvant, or other material mixed with the therapeutic as provided herein. In one embodiment, the therapeutic treats CDI. In one embodiment, the therapeutic prevents CDI.

Unlike the cell-based assays, which are the conventional and current standard for determining the presence of neutralizing antibody in a sample (e.g., serum, blood, or feces) of a subject that has or had CDI, the presently disclosed methods are amenable to packaging in a kit format, as discussed in more details below. This represents a significant distinguishing factor and a significant improvement over the current standard of assessment, as of the filing of this application. In particular, the cell-based approach required specialized equipment (e.g., microscopes) and expertise. Moreover, the cell-based methods were inherently subjective, as they rely on the accuracy of cell counts of an individual to suggest the presence or absence of neutralizing antibodies in a sample based on the observed shape of the cells in response to toxin. The cell-based approach was laborious and time consuming, requiring overnight incubation of the test cells. In contrast, the presently disclosed methods require little or no specialized equipment, provide rapid results (e.g., on the scale of an hour or less), and provide an objective measure of neutralizing antibodies in a sample that can be validated for mass commercial and clinical use. Altogether, the disclosed method specifically addresses a long-felt problem and enduring problem in the art by innovating a novel and unconventional approach.

### IV. Kits and Articles of Manufacture

In some embodiments, the underlying mechanism of this competition ELISA assay lies in the competitive binding of NAbs occurring in CDI patient blood samples that bind to major neutralizing epitopes on TcdB, the key virulence factor of C. diff.

The disclosed kits and articles of manufacture are in vitro diagnostic (IVD) tests for the semi-quantitative detection of Nab's directed to C. diff. TcdB in a biological sample (e.g., serum). In some embodiments, the kit can comprise a TcdB-coated microwell ELISA format where neutralizing anti-TcdB antibodies in patient serum compete for binding against neutralizing anti-TcdB antibodies. In some embodiments, the neutralizing anti-TcdB antibody is BB (BB-IgG). In some embodiments, the neutralizing anti-TcdB antibody is N2 (N3-IgG). In some embodiments, the neutralizing anti-TcdB antibody is N3 (N3-IgG). In some embodiments, the neutralizing anti-TcdB antibody is N11 (N11-IgG). In some embodiments, the neutralizing anti-TcdB antibody is bezlotoxumab. BB-IgGs recognizes major immunodominant neutralizing epitopes on TcdB.

In one exemplary use of the disclosed kits and articles of manufacture, human serum samples and biotinylated BB-IgGs are incubated in wells of TcdB -coated microwell plate allowing for competition of binding to immobilized TcdB. The antibodies against the major neutralizing epitopes on TcdB in patient serum sample compete against binding of BB-IgGs to the immobilized antigen and unbound biotinylated BB-IgGs is removed by wash steps. The remaining bound biotinylated BB-IgGs is detected by addition of a horseradish peroxidase-conjugated (HRP) conjugated streptavidin, which negatively correlate to the levels of patient neutralizing anti-TcdB titers. A color development reaction is utilized for optical absorbance measured by a standard ELISA microplate reader. Different shades of color correspond to different levels of anti-TcdB neutralizing antibodies present in the test serum. The deeper the color, the less bound NAb titers against TcdB. If the patient's NAb titers against TcdB are found to be below a threshold, then the patient is at a higher risk of recurrence and treatment with an anti-toxin (e.g. (ZINPLAVA™ (bezlotoxumab))) should be considered (FIG. 8). In some embodiments, the ELISA turn-around-time is about 15 minutes, about 30 minutes, about 45 minutes, about 1 hour, about 1 hour and 15 minutes, about 1 hour and 30 minutes, about 1 hour and 45 minutes, or about 2 hours. In some embodiments, the ELISA turn-around-time is at least 30 minutes. In some embodiments, the ELISA turn-around-time is at least 1 hour. In some embodiments, the ELISA turn-around-time is 1 hour.

As noted above, when a peroxidase, such as HRP is utilized as the label for the labeled antibody, the corresponding kits may also include a chemical substrate (be it chromogenic, fluorescent, chemiluminescent, or colorimetric) on which the enzyme can act. Non-limiting examples include 3,3′,5,5′-Tetramethylbenzidine (TMB), hydroxypheny-lacetic acid, 3-p-hydroxyphenylproprionic acid (HPPA), luminol, polyphenols and acridine esters, and luciferin. Thus, in some embodiments, kits which utilize an enzyme as the label can optionally comprise a chemical substrate, such as 3,3′,5,5′-Tetramethylbenzidine (TMB), hydroxypheny-lacetic acid, 3-p-hydroxyphenylproprionic acid (HPPA), luminol, a polyphenol, an acridine ester, or luciferin.

In certain embodiments, the present disclosure provides kits for performing the methods of this disclosure as well as instructions for carrying out the methods of the present disclosure. The kit comprises, or alternatively consists essentially of, or yet further consists of one or more of: a Clostridioides difficile (C. diff) toxin B (TcdB)-coated substrate; and a labeled antibody that binds to TcdB. In one embodiment, the kit includes a TcdB-coated plate, a biotinylated BB-IgG, and streptavidin-labeled horseradish peroxidase (HRP). In one embodiment, the kit further comprises unlabeled BB-IgG. In one embodiment, the kit further comprises an unlabeled antibody that binds to TcdB. In one embodiment, the unlabeled antibody is the same or different from the labeled antibody that binds to TcdB. In one embodiment, the labeled antibody comprises a label selected from a tag, a fluorophore, an enzyme, a gold particle, a magnetic particle, a dye, or a radiolabel/isotope.

As amenable, these disclosed kit components may be packaged in a manner customary for use by those of skill in the art.

### EXAMPLES

These Examples are provided for illustrative purposes only and not to limit the scope of the claims provided herein.

### Example 1: Evaluating a Panel of Neutralizing Anti-TcdB Antibodies in a Competition ELISA Assay

A panel of human neutralizing anti-TcdB antibodies was generated. The panel included the anti-TcdB antibodies in FIG. 5 and antibodies from an immuned library of alpacas through phage display and screening. As some of these neutralizing antibodies target broadly neutralizing epitopes, the antibodies specificity was evaluated and the correlation of the ELISA readings with anti-TcdB neutralizing titers in patient sera was compared. The neutralizing activities of these antibodies against toxins from a wide variety of C. diff. clinical isolations was also evaluated. After additional bNAbs are identified, we will combine these with N3 in competition ELISA assay.

Additional neutralizing anti-TcdB antibodies generated from patients and animals were evaluated. The neutralizing anti-TcdB antibodies N2 and N11 are human IgG1s; E3, 5D, 2Ds, 2D, and 7F are V_{H}Hs generated from TcdB (a glucosyltransferase-deficient holotoxin B)-immunized alpacas through phage display. All these antibodies bind to epitopes different from that of N3 See Yang et al, (2014) JID 2014:210. E3 binds to the glucosyltransferase domain of TcdB and broadly neutralizes TcdB from all tested normal pathogenic strains that produce both TcdA and TcdB, except for a TcdB variant from a divergent ribotype 17 strains. 7F binds to the autoprocessing site of TcdB and blocks cysteine protease (CPD)-mediated autocleavage of TcdB 61.

The broadly neutralizing activities of the antibodies identified against toxins from a wide variety of C. diff. clinical isolations were determined. Functional analyses of the neutralizing anti-TcdB antibodies was performed to test their abilities to broadly neutralize toxins from clinically relevant isolates. The neutralizing activity of a bi-specific antibody against the toxins from 13 C. diff. strains has been previously determined. This panel of C. diff. clinical isolations represents an assortment of genetically and geographically diverse clinical isolates. The bacterial culture supernatants were collected from each strain and TcdA was neutralized using the specific neutralizing antibodies. In addition, Vero cells (ATCC, Manassa, VA) that are highly sensitive to TcdB, but relatively insensitive to TcdA-induced cell rounding were used. The treated supernatants were mixed with the individual antibodies identified before being applied to the Vero cells. Cell rounding was monitored. In addition to these 13 strains that we have already cultured in the lab, we obtain additional strains collected by CDC (https://www.beiresources.org/Home.aspx) with ribotypes that are not covered in the previous collections to validate the bNAbs against.

### Example 2: Investigate Utility of bNAbs-Based Competition ELISA to Measure Neutralizing Anti-TcdB Titers in Serum Samples from CDI Patients

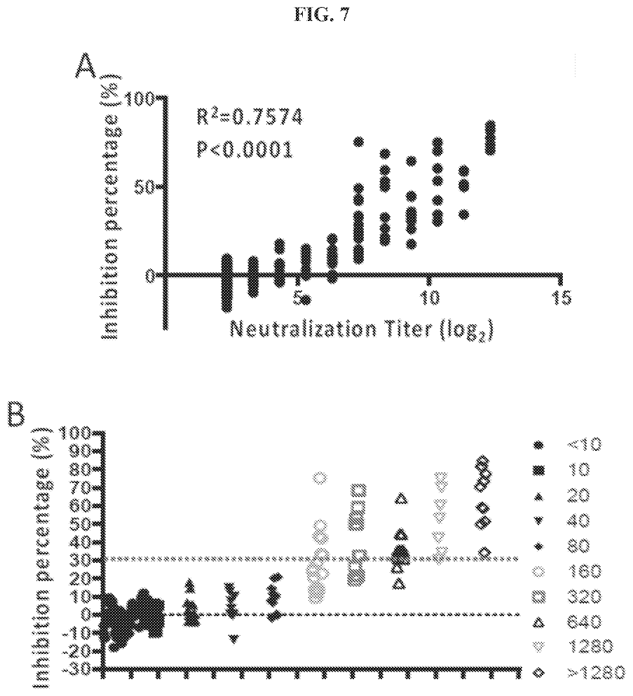

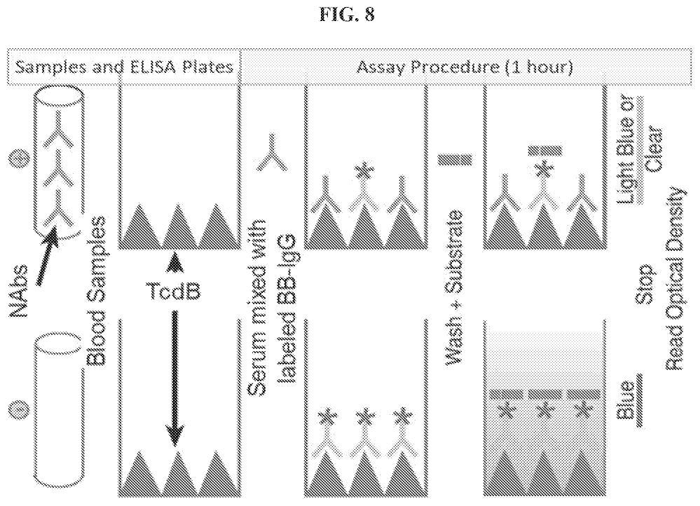

Several broadly neutralizing antibodies (bNAb) were identified against TcdB (N2, N3, N11 and BB). The bNAb's were biotinylated and a competition ELISA was developed. Using patient serum, the ELISA readings correlated with the patients' anti-TcdB neutralizing titers. The anti-TcdB neutralizing titers for each sample are measured by cell-based assays (FIG. 7A) the BB-IgG1 based competition ELISA (FIG. 7B). Specifically, the samples were coded and the information of the neutralizing titers was available only after the ELISA testing. The BB-IgG is most potent candidate for developing the assay in the present disclosure as seen in FIG. 6. The BB antibody was selected for further testing with a large scale sample size and the FZata C. Diff NAb Detect™ IgG competitive ELISA kit (BB-ELISA Kit) was developed. To validate the kit, 147 banked serum samples from CDI patients with known recurrence outcomes were selected and tested. The percentage of inhibition of anti-TcdB titers is provided in FIG. 7. The BB-ELISA Kit has comparative results compared to the cell-based assays. This demonstrates an unconventional technical solution that solves the technical problem in the field.

### Example 3: Preparation and Components of the BB-ELISA

The recombinant TcdB is generated from Bacillus megaterium according to Yang 2008 BMC Microbiology. Cultured B. megaterium is harvested and lysed and TcdB is purified by His-tag chromatography. BB-IgG1 is purified from HEK293 culture supernatant (freedom HEK from ThermoFisher by transient transfection) using a protein A column and biotinylated as described in Example 4. The kit comprises TcdB coated ELISA plates, Biotinylated BB-IgG1 in buffer, Streptavidin-HRP, BB-IgG1 (spiked in sera) as positive control/calibrator, and substrates.

### Example 4: Biotinylation of BB-IgG

Aliquots of purified TcdB stored at −80° C. were thawed on ice. Immuno Nonsterile 96-well plates or 384-well plates were coated with TcdB in 1×PBS buffer (pH 7.4) at lug/ml by dispensing or 25 ul using Rainin liquid handling system. The plates were tapped to ensure the coating antigen covered the whole bottom of the well. The plates were sealed with a sticky plastic cover and placed at 4° C. overnight. The coated plates were uncovered and placed on Microplate Washer and washed for 1 cycle, 4×/cycle and then tapped to dry. 2.5% skinny milk in PBS was balanced to room temperature and loaded at 200 ul/well for 96-well plate, or 100 ul/well for 384-well plate and incubated at room temperate for 1 hour for blocking. The samples, positive and negative controls were 5× diluted. Biotinylated-TcdB was used at a final concentration of 100 ng/ml in 2.5% of milk in PBS. The positive control was 100 ng/ml of biotinylated-TcdB in 2.5% of milk in PBS. Negative control was 2.5% of milk in PBS only. The CDI patient serum samples and aliquots of biotinylated-TcdB are taken were thawed on ice. Once the serum thawed completely, serum samples were 1: 2.5× diluted in 5% skinny milk in PBS. Biotinylated-TcdB was prepared at 400 ng/ml in 1×PBS buffer (PH 7.4). Testing samples were mixed with 1: 2.5× diluted serum sample and 400 ng/ml biotinylated-TcdB at 1:1 ratio (V/V). Positive control was prepared by mixing 400 ng/ml biotinylated-TcdB with 1×PBS buffer (PH 7.4) at 1:1 ratio (V/V). Positive control was mixed with 5% skinny milk in PBS and 1×PBS buffer (PH 7.4) at 1:1 ratio (V/V). After 1 hour of blocking, the plates were washed on Microplate Washer for 1 cycle, 4× wash/cycle and tapped to dry. The samples and controls were loaded 50 ul/well (96 well plates) or 25 ul/well (384-well plates). The plates stand at 37° C. for 1 hour incubation. After 1 h incubation, plates were washed on Microplate Washer for 1 cycle, 4× wash/cycle and tapped dry. Pre-diluted Pierce™ High Sensitivity Streptavidin-HRP was prepared at 1:100× in 2.5% of skinny milk in PBS according to the product description. Detecting antibody was added at 50 ul/well for 96-well plates or 25 ul/well for 384-well plates. Plates were incubated at room temperate for 1 hour. TMB 2-Component Microwell Peroxidase Substrate Kit was balanced at RT. Mixed at 1:1 ratio (V/V). Substrate was at 50 ul/well for 96-well plates or 25 ul/well for 384-well plates and incubated at room temperate within 15 minutes to avoid light for color development. Sulfuric Acid, 1 N, was added to each well (50 ul/well for 96-well plates or 25 ul/well for 384-well plates) to stop reaction. ELISA signals were read on Cytation™ 3, BioTek at OD450 nm within 30 min.

### Example 5: Use of BB-HRP to Replace Biotinylated BB-IgG1 and Avidin/Streptoavidin-HRP Conjugate

BB-HRP (5D-E3-conjugated with horseradish peroxidase (HRP) or E3-5D- HRP) is a neutralizing anti-TcdB bi-specific domain antibody fused with HRP via GS flexible linker. This fusion protein can be expressed bacteria, yeast, or mammalian cells and purified by an affinity chromatography. 5D-E3 can be either wild type V_{H}Hs or humanized versions. Humanized 5D-E3 has comparable binding and neutralizing activity as wild type version. When in use, BB-HRP will be mixed with the tested biological sample for 1 h of incubation and washed, then the substrate will be directly added to wells for color developing. Compared to biotinylated-BB-IgG1, BB-HRP will reduce the steps and duration of the assay, allowing a rapid BB-ELISA assay within 1 hr.

### Example 6: Data Analysis of the Competitive ELISA Results

All duplicates should exhibit similar OD 450 nm reading and CV within 20%. Positive controls should exhibit highest OD 450 nm reading. Negative controls should have OD 450 nm reading around 0.05. Most serum samples should show OD450 nm reading between positive controls and negative controls. Inhibition % of serum sample was calculated using the formula: Inhibition %=(1−mean of OD450 nm of sample/mean of OD450 nm of positive control)×100.

The OD450 nm data is copied to the BB-competitive ELISA template sheet. The QC and CV will be calculated and the BB inhibition results will be generated. The data is matched against the data interpretation table which is based on the data information collected both from the laboratory of neutralizing titer, competitive BB-inhibition clinical analysis of disease severity. The table explains the level of inhibition present, the level of disease severity, and the risk of developing severe disease.

### Example 7: Validation of the Standardized BB-ELISA for Measuring Anti-TcdB Neutralizing Activities in CDI Patient Sera

The BB-ELISA kit is used to measure anti-TcdB neutralizing activities in CDI patient serum samples, and the results are analyzed for correlation with neutralizing titers measured using cell-based assay. The utility of the BB-ELISA kit for predicting CDI disease severity and recurrence evaluated against approximately 300 serum samples of CDI patients with clear clinical history of CDI. The data is analyzed for correlation of recurrence and disease prediction. The primary endpoint is the correlation of the results with recurrence; the secondary endpoint is the correlation with disease severity progression. A low cutoff for positive prediction value (PPV) is established and is indicative of a high likelihood (i.e. 70% possibility) of recurrence. A high cutoff for negative prediction value (NPV) is established and indicates indicate unlikelihood (i.e. 5% possibility) of recurrence. Additional confounding factors are considered such as Age, underlying diseases, disease severity, immune status (immunocompetent/immunocompromised), days of blood drawing post a positive diagnosis, and primary or recurrent diseases.

### Example 8: Data Analysis of the Competitive ELISA Results

The OD450 nm data is copied to the BB-competitive ELISA template sheet. The QC and CV will be calculated and the BB inhibition results will be generated. The data is matched against the data interpretation table which is based on the data information collected both from the laboratory of neutralizing titer, competitive BB-inhibition clinical analysis of disease severity. The table explains the level of inhibition present, the level of disease severity, and the risk of developing severe disease.

### Example 9: BB-ELISA: Biotin-BB-IgG Competitive ELISA to Detect Neutralizing Anti-TcdB Antibody Response in Clostridioides difficile Infection (CDI) Patients

Aliquots of purified TcdB stored in −80° C. were thawed on ice. Immuno Nonsterile 96-well plates were coated in 1 ug/ml of TcdB in 1×PBS buffer (pH 7.4) by dispensing 50 ul using Rainin liquid handling system. The pates were tapped to ensure the coating antigen covered the whole bottom of the well. The plates were sealed with a sticky plastic cover and place at 4 OC overnight. On the second day, the coated plates were uncovered and placed on a Microplate Washer and washed for 1 cycle, 4×/cycle and tapped dry. The plates were blocked with 2.5% skinny milk in PBS balanced to room temperature by loading 200 ul/well to 96-well plates and incubated for 1 hour at room temperature. While the plates are blocking, the samples, positive, and negative controls were prepared. The internal positive control was 200 ng/ml Bio-BB-IgG in 2.5% skinny milk in PBS. The internal negative (blank) control was 2.5% skinny milk in PBS. The serum samples (5× dilution in final solution) were mixed with 200 ng/ml of Bio-BB-IgG in 2.5% skinny milk in PBS. After blocking, the plates were washed on a Microplate Washer for 1 cycle, 4× wash/cycle and tapped dry. 50 ul/well of the prepared samples, negative control and positive control were loading into the plates in duplicates. The plates stand at 37 OC for 1 hour of incubation. After incubation, the plates were washed on a Microplate Washer for 1 cycle, 4× wash/cycle and tapped dry. The pre-diluted (100×) Pierce™ High Sensitivity Streptavidin-HRP 100× was further diluted in 2.5% of skim milk in PBS according to the product description. 50 ul/well of the Streptavidin-HRP solution was added to the plates. The plates were incubated at room temperature for 1 hour. After incubation, the plates were washed on a Microplate Washer for 2 cycles, 4× wash/cycle and tapped dry. The TMB 2-Component Microwell Peroxidase Substrate Kit was balanced at room temperature and mixed at 1:1 ratio (V/V). The mixed substrate solution was added at 50 uL per well to the 96-well plates and incubated at room temperature within 5-15 min avoid of light for color development. Sulfuric Acid, 1 N, was added to each well at 50 ul/well to stop the reaction. The ELISA signals were read on Cytation™ 3, BioTek at OD450 nm within 30 minutes.

### Results Analysis

All duplicates should exhibit similar OD 450 nm reading and CV within 20%. The positive controls should exhibit highest OD 450 nm readings and the negative controls should have OD 450 nm reading around 0.05. Most serum samples should show OD450 nm reading between positive controls and negative controls. The percentage of Inhibition by serum sample was calculated using the formula: Inhibition %=(1−mean of OD450 nm of the sample/mean of OD450 nm of positive control)×100.

Unless otherwise defined, all technical and scientific terms used herein have the same meaning as commonly understood by one of ordinary skill in the art to which this technology belongs.

The present technology illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the present technology claimed.

Thus, it should be understood that the materials, methods, and examples provided here are representative of preferred aspects, are exemplary, and are not intended as limitations on the scope of the present technology.

The present technology has been described broadly and generically herein. Each of the narrower species and sub-generic groupings falling within the generic disclosure also form part of the present technology. This includes the generic description of the present technology with a proviso or negative limitation removing any subject matter from the genus, regardless of whether or not the excised material is specifically recited herein.

In addition, where features or aspects of the present technology are described in terms of Markush groups, those skilled in the art will recognize that the present technology is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

Other aspects are set forth within the following claims.

1. A method of detecting antibodies that bind to Clostridioides difficile (C. diff) toxin B (TcdB) in a biological sample, comprising:
(a) contacting a substrate to which TcdB is attached with (i) a biological sample and (ii) a labeled antibody that binds to TcdB,
(b) washing the substrate, and
(c) detecting a signal from the labeled antibody, wherein the amount of the signal detected is inversely correlated to the amount of antibodies that binds to TcdB in the sample;
wherein the biological sample was obtained from a subject that had a Clostridioides difficile infection (CDI) prior to obtaining the sample.

2. The method of claim 1, wherein the amount of signal is correlated against a predetermined threshold.

3. The method of claim 1, wherein the predetermined threshold is determined by an aggregate of clinical samples.

4. The method of claim 3, wherein the clinical samples comprises at least about 50 clinical samples.

5. The method of claim 1, wherein the labeled antibody is a neutralizing anti-TcdB antibody.

6. The method of claim 5, wherein the labeled antibody is selected from ZINPLAVA™ (bezlotoxumab)) N2-IgG, N3-IgG, N11-IgG, C6-IgG, C12-IgG, 2D-IgG, 5D-IgG, E3-IgG, 7F-IgG, A1-IgG, A11-IgG, B7-IgG, B12-IgG, or BB-IgG.

7. The method of claim 1, wherein the antibody that binds to TcdB in the sample is a neutralizing anti-TcdB antibody.

8. The method of claim 1, wherein the biological sample is selected from whole blood, isolated blood cells, plasma, serum, feces, or urine.

9. The method of claim 1, wherein the labeled antibody comprises a label selected from a tag, a fluorophore, an enzyme, a gold particle, a magnetic particle, a dye, or a radiolabel/isotope.

10. The method of claim 9, wherein the tag is biotin or epitope tag.

11. The method of claim 1, wherein the signal is selected from fluorescence, electrochemical, chemiluminescence, or bioluminescence.

12-22. (canceled)

23. A method of determining likelihood of Clostridioides difficile infection (CDI) recurrence or prognosing CDI, comprising:
(a) contacting a substrate to which Clostridioides difficile (C. diff) toxin B (TcdB) is attached with (i) a biological sample and (ii) a labeled antibody that binds to TcdB,
(b) washing the substrate, and
(c) detecting a signal from the labeled antibody, wherein a signal intensity that is below a predetermined threshold is indicative of (i) recurrent CDI or a risk thereof, or (ii) a CDI severity or a risk of recurrent CDI, and a signal intensity that is above the predetermined threshold is indicative of lack of CDI severity or a low risk of recurrent CDI;
wherein the biological sample was obtained from a subject that had or has a Clostridioides difficile infection (CDI).

24-33. (canceled)

34. A method of treating Clostridioides difficile infection (CDI) in a subject, comprising:
(a) obtaining a biological sample, wherein the biological sample was obtained from a subject that had a Clostridioides difficile infection (CDI) prior to obtaining the sample,
(b) contacting a substrate to which TcdB is attached with (i) the biological sample and (ii) a labeled antibody that binds to TcdB,
(c) washing the substrate,
(d) detecting a signal from the labeled antibody, wherein the amount of the signal is inversely correlated to CDI severity or likelihood of development of recurrent CDI, and
(e) treating the subject with a therapeutic for CDI if the amount of the signal is below a predetermined threshold.

35. The method of claim 3 wherein the subject is currently being treated with antibiotics, and halting treatment of the subject with antibiotics if the amount of the signal is above a predetermined threshold.

36-48. (canceled)

49. A kit comprising:
(a) a Clostridioides difficile (C. diff) toxin B (TcdB)-coated substrate; and
(b) a labeled antibody that binds to TcdB; or
a TcdB-coated plate, an enzyme-labeled BB-IgG, and a chemical substrate for the enzyme, wherein the labeled BB-IgG comprises horseradish peroxidase (HRP) conjugated directly or indirectly to BB-IgG.

50-60. (canceled)
